(19) United States
(12) Patent Application Publication
(10) Pub. No.: US 2005/0090721 A1
(43) Pub. Date: Apr. 28, 2005

(54) Weighing and pump system for a bed

(76) Inventor: Shahzad Pirzada, Manhasset, NY (US)

Correspondence Address: WILLIAM COLLARD;COLLARD & ROE, P.C., 1077 NORTHERN BOULEVARD, ROSLYN, NY, 11576, US

(21) Appl. No.: 10/939,816

(22) Filed: Sep. 13, 2004

Related U.S. Application Data
(63) Continuation-in-part of application No. 10/162,279, filed on Jun. 4, 2002, now Pat. No. 6,789,283.
(63) Continuation-in-part of application No. 09/812,166, filed on Mar. 19, 2001, now Pat. No. 6,564,411.

(51) Int. Cl. A61B 5/00

(52) U.S. Cl. 600/300

(57) ABSTRACT

A portable patient management system which can be used to monitor the particular health characteristics of a patient. This device can include a central housing, a pump disposed in the central housing and at least one display disposed in the central housing. To control this pump there is at least one processor disposed in the central housing. This processor is in communication with the pump and the display. There is also at least one memory unit disposed in the central housing. This memory unit can contain a plurality of instructions to control the processor. There is also at least one communication element disposed in the central housing. The communication element allows the central housing to communicate with other components.

### CROSS REFERENCE TO RELATED APPLICATIONS

This application is a continuation in part application and hereby claims priority under 35 U.S.C. 120 from U.S. patent application Ser. No. 10/162279 filed on Jun. 10, 2002 which also claims priority under 35 U.S.C. 120 from U.S. patent application Ser. No. 09/812,166 filed on Mar. 19, 2001 now issued as U.S. Pat. No. 6,564,411 wherein the disclosures of both of these references are hereby incorporated herein by reference.

### BACKGROUND OF THE INVENTION

The invention relates to a pump which can be used to provide support for a bed. This pump is in the form of a portable pump that can be in the form of a wireless pump. In addition the invention can also relate to a remote weighing system that can be integrated with a pump and a central patient management system for activating or deactivating a pump based upon a weight registered by the remote weighing system.

### SUMMARY OF THE INVENTION

The invention relates to a portable patient management system which can be used to monitor the particular health characteristics of a patient. This device can include a central housing, a pump disposed in the central housing and at least one display disposed in the central housing.

To control this pump, there is at least one processor disposed in the central housing. This processor is in communication with the pump and the display.

There is also at least one memory unit disposed in the central housing. This memory unit can contain a plurality of instructions to control the processor. There is also at least one communication element disposed in the central housing. The communication element allows the central housing to communicate with other components.

The other components can include a remote scale, having its own housing and a remote communication element stored in housing, this remote communication element can be for communicating with the at least one communication element, to transmit a weight measurement to the central measurement device.

### BRIEF DESCRIPTION OF THE DRAWINGS

Other objects and features of the present invention will become apparent from the following detailed description considered in connection with the accompanying drawings. It should be understood, however, that the drawings are designed for the purpose of illustration only and not as a definition of the limits of the invention.

In the drawings, wherein similar reference characters denote similar elements throughout the several views:

FIG. 1 is a perspective view of the system including an air mattress;

FIG. 2 is a front view of the central housing revealing the components disposed in the central housing;

FIG. 3A is a side view of a remote scale; and

FIG. 3B is a top view of the remote scale shown in FIG. 3A.

### DETAILED DESCRIPTION OF PREFERRED EMBODIMENTS

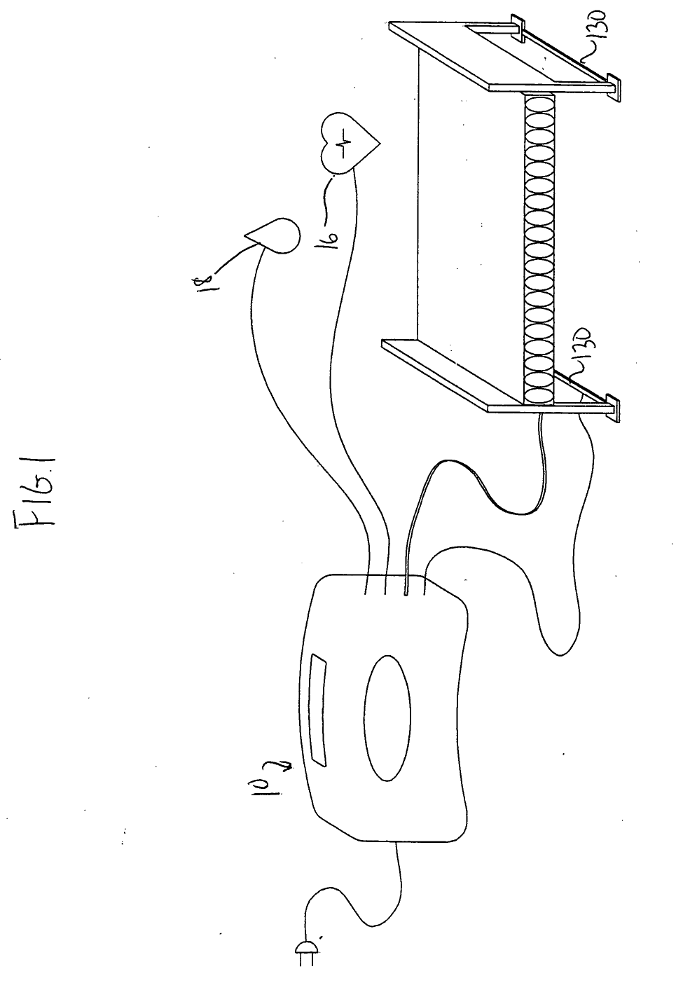

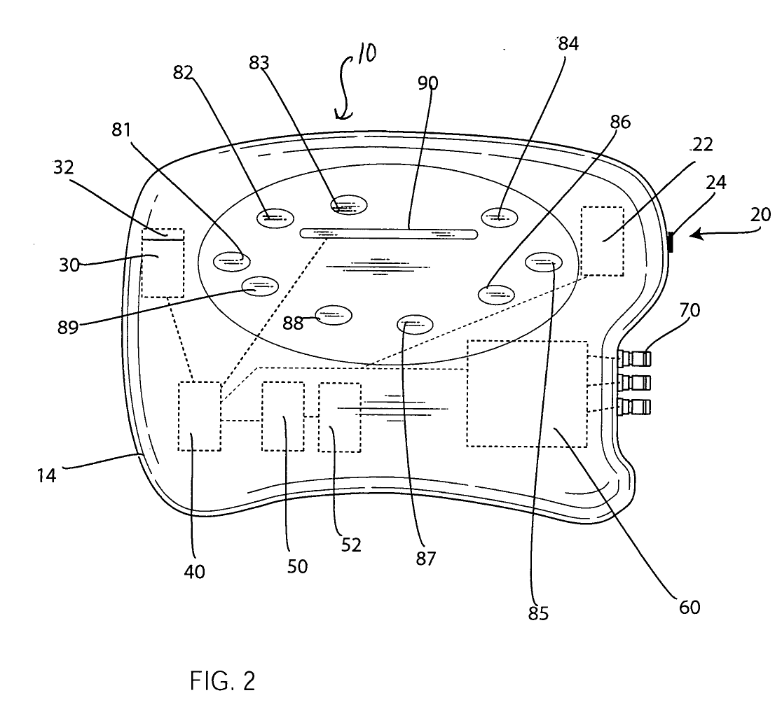

Turning now in detail to the drawings, FIG. 1 shows an overview of a patient management system 10. This patient management system 10 includes a central patient measurement device 12 including a central housing 14. This central patient measurement device can include a communication element 20 (See FIG. 2) for wired or wireless communication with adjacent remote components. This communication element may contain a wireless transceiver 22 or a hard wired ethernet connection 24 for connecting an ethernet cable to housing 14. In addition, coupled to housing 14 can be additional patient monitoring devices such as a blood pressure monitor 16 or a temperature monitor 18. Temperature monitor can be in the form of a strap on thermometer via a pad incorporated into a bed or via any other known thermometer or temperature taking device. Other known thermometer devices are known in the art such as in U.S. Pat. No. 6,454,724 incorporated herein by reference. Additional measuring devices may be coupled to housing 14 and in communication with the patient measuring device as well.

FIG. 2 shows the device 12 which has numerous components stored inside of a housing 14. Housing 14 can be made from any durable or semi-durable material such as steel, aluminum or plastic. In this case, these components include a power source 30 which can include a battery 32, a processor 40, a memory 50, an optional second memory 52, and a pump 60. In addition, coupled to this housing are valves 70 which can be used for fluid intake and for fluid outflow wherein the fluid can be for example, air. A timer 76 is disposed in housing 14 and can be used to regulate particular processes such as a time for reading a weight of a user or a time for starting pump 60. Disposed on an external surface of the pump is a set of keys or buttons 80 for receiving information from a user and also a display 90 for displaying information. Display 90 can be in the form of a 16 character dot matrix LCD display which can be used to display either pounds (LBS) or kilograms (KG).

With this design, power source 30 can include an outlet or plug to an external power source such as a wall outlet producing 110 or 220 VAC at approximately 50-60 Hz. Other known power sources can also be used as well. In addition, this power source 30 is in electrical communication with battery 32 and can be used to recharge a battery 32. Power source 30 is used to power the remaining components in the housing.

Processor 40 is in communication with, and can be used to control the remaining components in housing 14. For example, processor 40 receives instructions from a memory 50 and also input keys 80 wherein this processor processes these instructions to perform particular tasks such as instructing pump 60 to pump additional fluid into a remote compartment such as an air bed.

In addition, processor 40 can also process any information received from communication device 20, to create a readout on display 90 to instruct users on a patient's weight or other vital statistics.

Device 12 can be in communication via communication device 20 with a remote scale 130, which in this embodiment is in the form of a bar which stretches across two legs of a bed 140. In this case, this remote scale can be used to measure the weight of a bed and also the weight of a user on a bed. To determine the weight of a user on a bed, the scale can first be calibrated to determine the weight of the bed. The information on the weight of bed 140 is received by communication device 20 either through wired commination through communication port 24 or through wireless communication through wireless transceiver 22. This information is next processed by processor 40 and then the resulting weigh is displayed on display 90.

Once the weight of the bed has been determined, the weight scale can be synchronized to zero out, to create an effective weight of zero. A zero key 82 can thereby be pressed to zero out the total weight calculated to this point. This device can also be used to determine a users vital statistics such as temperature or blood pressure. For example, there is a temperature button 81 and a blood pressure button 83. When a user takes a patient's temperature and presses temperature button 81, display 90 then reads the temperature of the patient. In addition, when a user takes a blood pressure reading of a patient, and presses blood pressure button 83 display 90 would then render a blood pressure reading for the user as well.

Next, as the user is placed on the bed, the total weight is determined at this new zeroed out setting to determine the weight of the user.

This device 12 can also be used to determine the effective force or weight on multiple different scales and then either average this weight to arrive at an overall weight or report on each individual weight as well. Thus, there is a cycle weight button 84 which can be used to allow a user to cycle through various weights for a user such as an overall average weight or a weight for each one of the scales. In addition, there is an auto zero button 85 which allows the user to zero the scale. Furthermore, there is also a hold or freeze/resume button 86 which can be used to freeze and store any displayed weight. In this case, the stored weight can be stored in memory 50 or in additional memory 52. This freeze/resume button 86 allows the user to freeze the patients weight in the readout and then make any subsequent changes to an adjacent bed before resuming measurements.

A weight recall button 87 can also be used to display on display 90 a last known or recorded weight. This information may be stored in memory 50 or additional memory unit 52. A weight change button 88 can also be used to present to a user the difference in weight of a patient across a control group. In this case, a user can set an initial weight so that a subsequent weight change can be recorded or, processor 40 and memory 50 can record a weight change across a period of time such as a day, a week, a month or a year. Finally, a weight toggle button 89 can be used to toggle between pounds and kg in the readout of display 90.

Memory unit 50 and memory unit 52 can be in the form of a flash memory, a form of random access memory or RAM, or any other memory unit known in the art. These memory units can hold their memory even if power is lost. This device can operate with one single memory unit 50 or with an additional memory unit 52. In one embodiment, memory unit 50 can be used to store a set of instructions, operating system or program to control processor 40 and the remaining components. Additional memory unit 52 can therefore be used to store a patient's information or vital statistics such as weight, blood pressure, respiratory results, temperature etc.

With these buttons it is possible to easily move a bed and a patient without losing the settings of a patient. In this case, before moving the bed or the patient, the user can press the hold or freeze/resume button wherein display 90 will read “freeze”. Next, the user can make any necessary adjustments to the bed or patient such as moving the bed. Once the patient has been resettled, the user can then press the freeze/resume button again and resume normal weight monitoring.

If there is a loss of power, or if it is necessary to disconnect the bed power when the scale is in use, the user can press the freeze/resume button 86 and then resume the use of this device.

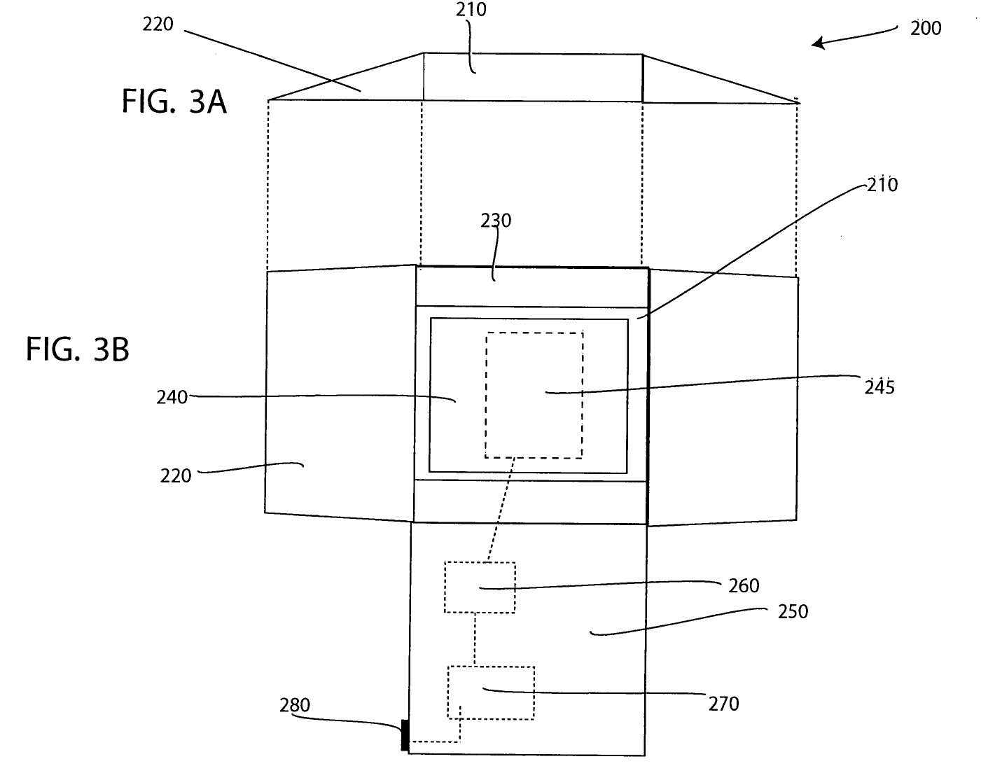

FIG. 3A shows a side view of a remote scale 200 which can include a central housing 210 which has ramps 220 disposed on either side. FIG. 3B shows a top view of this device wherein this device includes lateral supports 230 to support a wheel a bed leg or other similar type device on a pressure plate 240. These lateral supports are raised up above housing 210 to keep a wheel from rolling off. Plate 240 is disposed in housing 210 and is adjacent to, or on top of sensor 245. Sensor 245 can be in the form of a transducer such as a piezoelectric sensor. In addition, there is an adjacent housing 250, coupled to and disposed adjacent to housing 245. Disposed in adjacent housing 250 is a processor 260, to receive and process signals from sensor 245. These signals are then transmitted to an associated patient management system 10. Adjacent housing 250 can also include an additional or alternative communication connection 280 which can be in the form of an ethernet connection for communication via wires to hard wired ethernet connection 24.

With this design, a user can place one or more of these remote scales 200 in a position adjacent to a bed. In many cases, the bed can be transportable on wheels. This bed can then be rolled up on ramps 220 so that wheels on the bottom of a bed frame rest on impression or pressure plate 240. The weight of this bed then presses down on pressure plate 240 to provide a pressure on sensor 245. The signals based on the weight pressure on sensor 245 are then transmitted to processor 260 wherein these signals are then transformed into communicatable signals via wireless transmitter/transceiver 270 or through wired ethernet connection 280.

Accordingly, while a few embodiments of the present invention have been shown and described, it is to be understood that many changes and modifications may be made thereunto without departing from the spirit and scope of the invention as defined in the appended claims.

1. A portable patient management system which can be used to monitor the particular health characteristics of a patient comprising:
a) a central housing;
b) a pump disposed in said central housing;
c) at least one display disposed in said central housing;
d) at least one processor disposed in said central housing said processor in communication with said pump and said display;
e) at least one memory unit disposed in said central housing for containing a plurality of instructions to control said processor;
f) at least one communication element disposed in said central housing;
d) at least one remote scale, having a housing and a remote communication element stored in said housing, said remote communication element for communicating with said at least one communication element, to transmit a weight measurement to said processor in said central housing.

2. The system as in claim 1, wherein said at least one remote scale has a sensor, and wherein said housing on said at least one remote scale includes at least one ramp, and at least one impression plate disposed in said housing adjacent to said ramp, said impression plate for allowing a device having a weight to press against said sensor.

3. The system as in claim 1, wherein said sensor is a transducer.

4. The system as in claim 1, wherein said sensor is a piezoelectric sensor.

5. The system as in claim 1, wherein said sensor is in the form of a leaf spring which when flexed provides a measurement for a weight of an element to be weighed.

6. The system as in claim 1, wherein said housing for said remote scale includes a lateral stabilizer for laterally stabilizing a device to be weighed.

7. The system as in claim 1, wherein said at least one remote scale further comprises a power unit disposed in said housing adjacent to said impression plate for providing power to said scale.

8. The system as in claim 2, wherein said sensor is disposed adjacent to said impression plate.

9. The system as in claim 8, wherein said sensor can be depressed by said impression plate when an object is placed on said impression plate.

10. The system as in claim 1, wherein said at least one communication element in said central housing includes a jack and wherein said communication element in said housing for said at least one remote scale includes a jack.

11. The system as in claim 10, wherein said at least one communication element is in the form of an ethernet connection.

12. The system as in claim 1, wherein said at least one communication element is in the form of a wireless communication element having a wireless transponder.

13. The system as in claim 12, wherein said at least one communication element for said at least one remote sensor is in the form of a wireless communication element having a wireless transponder.

14. The system as in claim 1, further comprising a pressure measuring device disposed in said central housing for measuring a gas pressure of an adjacent compartment.

15. The system as in claim 14, wherein said adjacent compartment is in the form of an air filled mattress.

16. The system as in claim 15, further comprising a conduit for fluid communication between said pressure measuring device and said adjacent compartment.

17. A portable patient management system which can be used to monitor the particular health characteristics of a patient comprising:
a) a central housing;
b) a pump disposed in said central housing;
c) at least one display disposed in said central housing;
d) at least one processor disposed in said central housing said processor in communication with said pump and said display;
e) at least one memory unit disposed in said central housing for containing a plurality of instructions to control said processor;
f) at least one wireless communication element disposed in said central housing;
g) pressure measuring device disposed in said central housing for measuring a gas pressure of an adjacent compartment;
h) at least one remote scale, having a housing and a remote wireless communication element disposed in said housing, said wireless remote communication element for communicating with said at least one wireless communication element, to transmit a weight measurement to said at least one central measurement device wherein said memory includes a plurality of instructions to control said processor and said pump to adjust a preset pressure range based upon a weight measurement in said at least one processor in said central housing.
